(19) United States
(12) Patent Application Publication
(10) Pub. No.: US 2023/0127548 A1
(43) Pub. Date: Apr. 27, 2023

(54) METHODS FOR IDENTIFYING A CELL UPTAKE MECHANISM

(71) Applicant: The Regents of the University of California, La Jolla, CA (US)

(72) Inventors: Eugene Yeo, La Jolla, CA (US); Eric Van Nostrand, La Jolla, CA (US); Nathan Gianneschi, La Jolla, CA (US); David A. Nelles, La Jolla, CA (US); Sarah Barnhill, La Jolla, CA (US); Anthony Rush, La Jolla, CA (US)

(21) Appl. No.: 17/936,216

(22) Filed: Sep. 28, 2022

Related U.S. Application Data
(60) Provisional application No. 63/249,892, filed on Sep. 29, 2021.

(51) Int. Cl. C12N 15/10

(52) U.S. Cl. CPC C12N 15/1079

(57) ABSTRACT

Provided herein are methods of identifying a cell uptake modulator of a molecule that include (a) contacting a plurality of cells of a cell-containing biological sample with a plurality of gene-editing agents, wherein a gene-editing agent from the plurality of gene-editing agents recognizes and alters a target gene of at least one cell of the plurality of cells; (b) contacting the plurality of cells with a plurality of molecules, wherein at least one molecule of the plurality of molecules is transported into at least one cell of the plurality of cells; and (c) detecting a presence of the at least one molecule in the plurality of cells, thereby identifying the cell uptake modulator of the molecule.

### CROSS-REFERENCE TO RELATED APPLICATIONS

This application claims priority to U.S. Provisional Application Serial No. 63/249,892, filed on Sep. 29, 2021. The disclosure of the prior application is considered part of the disclosure of this application, and is incorporated herein by reference in its entirety.

### SEQUENCE LISTING

This application contains a Sequence Listing that has been submitted electronically as an XML file named “15670-0347001.XML.” The XML file, created on Sep. 23, 2022, is 4,096 bytes in size. The material in the XML file is hereby incorporated by reference in its entirety.

### FEDERALLY SPONSORED RESEARCH OR DEVELOPMENT

This invention was made with Government support under Grant Nos. HG004659 and NS103172 awarded by the National Institutes of Health, and under DMR1710105 awarded by the National Science Foundation. The Government has certain rights in the invention.

### BACKGROUND

Current techniques and tools for nanocarrier and macromolecule development for therapeutic delivery are limited in scope and understanding of the processes involved in their uptake into target cells. This includes their active interactions with membrane transporters that co-ordinate cellular uptake and processing. Strategies to elucidate the mechanism of uptake, such as painstaking manipulation of individual effectors with pharmacological inhibitors or specific genetic knockdowns, are generally biased towards previously studied pathways or the intuition of the investigators. Furthermore, each of these approaches present significant off-target effects, clouding the outcomes. Therefore, methods for intracellular transport of nucleic acids are much sought after in the context of both in vitro delivery reagents and in vivo therapeutics.

### SUMMARY

The present disclosure is based, at least in part, on identifying a cell uptake modulator in a cell.

Provided herein are methods of identifying a cell uptake modulator of a molecule, the method comprising: (a) contacting a plurality of cells of a cell-containing biological sample with a plurality of gene-editing agents, wherein a gene-editing agent from the plurality of gene-editing agents recognizes and alters a target gene of at least one cell of the plurality of cells; (b) contacting the plurality of cells with a plurality of molecules, wherein at least one molecule of the plurality of molecules is transported into at least one cell of the plurality of cells; and (c) detecting a presence of the at least one molecule in the plurality of cells, thereby identifying the cell uptake modulator of the molecule.

In some embodiments, the gene-editing agent is a lentivirus. In some embodiments, the gene-editing agent comprises CRISPR/Cas9 components. In some embodiments, the gene-editing agent from the plurality of gene-editing agents comprises a single guide RNA (sgRNA) corresponding to a gene from a gene library, and wherein the plurality of gene-editing agents comprises different gene-editing agents comprising different sgRNAs from the gene library.

In some embodiments, the molecule is a DNA polymer micelle. In some embodiments, the DNA polymer micelle comprises a plurality of amphiphilic compounds, wherein the amphiphilic compound of the plurality of amphiphilic compounds comprises a hydrophobic polymer, a DNA sequence, and a detectable label. In some embodiments, the detectable label is a fluorophore. In some embodiments, the fluorophore is Cy5. In some embodiments, the detectable label is located at the 3′ end of the DNA sequence.

In some embodiments, the DNA sequence comprises about 30 nucleotides. In some embodiments, the DNA sequence comprises a spacer sequence.

In some embodiments, the DNA polymer micelle comprises about 20 amphiphilic compounds. In some embodiments, the DNA polymer micelle further comprises a therapeutic cargo. In some embodiments, the therapeutic cargo comprises a nucleic acid.

In some embodiments, the detecting step (c) further comprises: selecting a molecule uptake-deficient cell from the plurality of cells; isolating an sgRNA fragment from the molecule uptake-deficient cell; and profiling the isolated sgRNA fragment to identify the target gene of the isolated sgRNA fragment, wherein the target gene is the identified cell uptake modulator. In some embodiments, the profiling comprises sequencing of the isolated sgRNA fragment. In some embodiments, the sequencing comprises high-throughput sequencing.

In some embodiments, the identified cell uptake modulator is SLC18B1. In some embodiments, the cell-containing biological sample comprises a tissue sample. In some embodiments, the cell-containing biological sample comprises live cells from a cell culture.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1E show improved library preparation for CRISPR/Cas9 pooled screens. FIG. 1A shows in pooled CRISPR/Cas9 screening, first a pool of lentivirus is made in which each lentiviral particle contains sgRNAs targeting one of hundreds to thousands of genes. After infection at low multiplicity of infection to ensure at most one lentiviral particle infects each cell, cells are selected for viral infection for 10 days. In a simple screen for lethality, gDNA is then isolated and queried to look for sgRNAs which are no longer present (indicating that the targeted gene is essential). FIG. 1B shows RNA antisense probes were generated from PCR amplification of ~500 nt fragments of constant regions flanking the variable sgRNA region, followed by T7 in vitro transcription with biotinylated rUTP or rCTP. FIG. 1C shows to deplete non-sgRNA containing regions, genomic DNA is isolated and sonicated. Antisense probes are then bound to gDNA fragments, followed by purification with streptavidin beads. RNase treatment is then used to remove RNA probes and isolate ssDNA fragments containing sgDNA regions, followed by PCR to add adapters for high-throughput sequencing. FIG. 1D shows bar graphs that indicate enrichment performed on biological replicate Day 10 samples (A = ~4.4× 10⁶ cells, B = ~6.9× 10⁶ cells). Shown are input gDNA (dsDNA), supernatant (ssDNA), and pulldown (ssDNA) yields quantified by Nanodrop 2000. FIG. 1E shows a graph wherein points indicate (y-axis) yield (quantified by high-sensitivity D1000 tapestation) after 20 cycles of PCR amplification starting with (x-axis) indicated input gDNA amounts (or equivalent sample fraction for purified and supernatant samples).

FIGS. 2A-2B show improved library preparation for CRISPR/Cas9 pooled screens. FIG. 2A shows points that indicate gDNA yield per million HEK293T cells from 12 biological replicate experiments. Boxplot indicates 25th to 75th percentile, with median indicated.

FIG. 2B shows points that indicate (y-axis) DNA yield after 20 PCR cycles of amplification for (x-axis) various gDNA amounts. Shown are two biological replicates using Q5 2X master mix, Q5 Hot Start, and Herculase II Fusion DNA Polymerase PCR reagents.

FIGS. 3A-3H show reproducibility testing for improved library preparation for CRISPR/Cas9 pooled screens. FIG. 3A shows an exemplary schematic of infection and technical replicates to query experimental reproducibility. Coverage was extrapolated based on gDNA yield using standard 6.6 µg per 1 million cell estimates. FIG. 3B shows a heatmap indicating correlation (Pearson R) between reads-per-million normalized sgRNA counts (log2) from technical or biological replicate samples as indicated, using only sgRNAs with RPM>1 in at least one dataset. FIGS. 3C-3F show scatter plots indicating frequency of sgRNAs with indicated read coverage for (FIG. 3C) D10 100X coverage technical replicates, (FIG. 3D) D10 100X coverage infection replicates, (FIG. 3E) D10 50X coverage technical replicates, and (FIG. 3F) D10 25X coverage technical replicates.

FIG. 3G shows bars indicating correlation between replicates calculated for 65 replicate pairs from TKO library and 3 from Brunello library screens. Stars indicate pairwise correlations from FIG. 3B for FLI-seq infection replicates or technical replicate libraries.

FIG. 3H shows scatter plot indicating frequency of sgRNAs with indicated read coverage for an example replicate pair from TKO library screening. This pair had median correlation across the 68 pairings considered in FIG. 3G. For all, pearson correlation (R) indicated is calculated based only on sgRNAs with RPM>1 in at least one of the two datasets.

FIGS. 4A-4D show reproducibility testing for improved library preparation for CRISPR/Cas9 pooled screens. FIG. 4A shows cumulative distribution plots indicating fold-change between Day 0 and Day 10 observed for annotated essential genes, non-target controls, and all other genes. Significance was determined by Kolmogorov-Smirnov two-sided test. FIGS. 4B-4C show scatter plots indicating the density of sgRNAs showing the indicated normalized read coverage in (FIG. 4B) D10 technical replicates and (FIG. 4C) D10 infection replicates. See FIGS. 3C-3D for comparisons plotted on logarithmic scale.

FIG. 4B shows a heatmap indicating correlation (Pearson R) between reads-per-million normalized sgRNA counts from technical or biological replicate samples as indicated, using only sgRNAs with RPM>1 in at least one dataset. See FIG. 3B for correlations calculated on log2-transformed normalized read density.

FIGS. 5A-5G show screen for nanoparticle uptake that identifies SLC18B1 as a novel effector. FIG. 5A shows unimer and self-assembled structure of DPANPs. TEM of DPANPs shows discreet, ~20 nm micelles. Scale bar 100 nm. FIG. 5B shows an exemplary schematic of pooled screen. Whole-genome pooled sgRNA library lentivirus was transduced into HEK293T cells and viral infection was selected for 10 days with Puromycin. On Day 10, cells were treated with Cy5-labeled micelles, followed by FACS analysis to isolate the bottom ~2.5% fluorescent population as uptake-deficient. After library preparation, sgRNA enrichment in uptake-deficient versus unsorted population was used to identify candidate genes. FIG. 5C shows scatter plot indicating gene-level sgRNA enrichment (z-score) from two biological replicate screens performed with the Gecko “A” library. FIG. 5D shows bars indicating fluorescent micelle uptake in single-knockout validation experiments. Each bar indicates an independent sgRNA sequence, and error bars indicate standard deviation from replicate measurements. FIG. 5E shows bars indicating uptake observed in four clonal populations from SLC18B1 CRISPR/Cas9 knockout (with knockout confirmed by genomic PCR and sanger sequencing). FIG. 5F shows a Kaplan-Meier plot shows (y-axis) fraction of patients surviving for (x-axis) indicated time, with patients separated by high and low expression of SLC18B1 from TGCA data for head and neck cancer. Dotted lines indicate 95% confidence intervals, and censored datapoints are indicated by vertical lines. Plots were generated with KMplot package in MATLAB. FIG. 5G shows violin plots indicating expression of SLC18B1 observed across 54 tissues profiled by the GTEx consortium, with box plot indicating median and 25th to 75th percentile.

FIGS. 6A-6D show screen for nanoparticle uptake that identifies SLC18B1 as a novel effector. FIG. 6A shows from left to right: structure and sequence of DPANPs, pol = polymer. HPLC and corresponding mass (obtained via MALDI-TOF) of the Cy5 labeled DNA sequence. TEM micrograph of negatively stained DPANP micelles. PAGE gel of crude and purified DPANPs, visualized using ethidium bromide staining. FIG. 6B shows a Fluorescence Activated Cell Sorting (FACS) plot showing the regions selected as Cy5-low (putative uptake-deficient) and Cy5-high (putative uptake-increased) 293T cells that were infected with pooled CRISPR library. FIG. 6C show ranking of selected genes in initial analysis (z-score relative to non-targeting controls) and re-analysis with MAGeCK comparing Cy5-low versus unsorted cells. FIG. 6D shows a Kaplan-Meier plot shows (y-axis) fraction of patients surviving for (x-axis) indicated time, with patients separated by high and low expression of SLC18B1 from TGCA data for liver cancer. Dotted lines indicate 95% confidence intervals, and censored datapoints are indicated by vertical lines. Plots were generated with KMplot package in MATLAB.

### DETAILED DESCRIPTION

Methods for intracellular transport of nucleic acids are much sought after in the context of both in vitro delivery reagents and in vivo therapeutics. Recently, it was found that micellar assemblies of hundreds of amphiphiles consisting of single-stranded DNA which has been covalently linked to a hydrophobic polymer, referred to as DNA-polymer amphiphile nanoparticles or DPANPs, can readily access the cytosol of cells where they modulate mRNA expression of target genomes without transfection or other helper reagents, making them potential therapeutic nucleic acid carriers. However, despite their effective uptake properties and efficacy in the cytosol, it was unknown how these polyanionic structures can enter cells. Indeed, generally, bottlenecks in understanding and achieving delivery and uptake remain a forefront issue in translatability of macromolecular and nanomaterials-based therapeutics generally, including with respect to nucleic acid therapies. However, elucidating these mechanisms is typically done through a collection of poor, low throughput choices that include stepwise knockouts of genes postulated to be potential candidates (e.g. anion scavengers), the use of pharmacological inhibitors that often have broad side-effects, or membrane disruption methods that raise questions as to biological relevance. Thus, unbiased screening technologies for the identification of molecular regulators of uptake would dramatically improve the ability to develop and understand synthetic molecule and material uptake and transport.

Detailed herein are methods of identifying a cell uptake modulator of a molecule (e.g., micellar assemblies, DPANPs) in a cell. In some embodiments, a method of identifying a cell uptake modulator of a molecule can include (a) contacting a plurality of cells of a cell-containing biological sample with a plurality of gene-editing agents, wherein a gene-editing agent from the plurality of gene-editing agents recognizes and alters a target gene of at least one cell of the plurality of cells; (b) contacting the plurality of cells with a plurality of molecules, wherein at least one molecule of the plurality of molecules is transported into at least one cell of the plurality of cells; and (c) detecting a presence of the at least one molecule in the plurality of cells, thereby identifying the cell uptake modulator of the molecule.

Various non-limiting aspects of these methods are described herein, and can be used in any combination without limitation. Additional aspects of various components of methods for identifying a cell uptake modulator or cell uptake mechanism are known in the art.

It must be noted that, as used in the specification and the appended claims, the singular forms “a,” “an” and “the” include plural referents unless the context clearly dictates otherwise.

As used herein, “biological sample” can refer to a sample generally including cells and/or other biological material. A biological sample can be obtained from a mammalian organism. For example, a biological sample can be obtained from a human. A biological sample can be obtained from a non-human mammal (e.g., a dog, a cat, a monkey, a mouse, or a rat). A biological sample can be obtained from non-mammalian organisms (e.g., a plants, an insect, an arachnid, a nematode), a fungi, an amphibian, or a fish (e.g., zebrafish). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). Biological samples can be derived from a homogeneous culture or population of organisms or alternatively from a collection of several different organisms, for example, in a community or ecosystem.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

In some embodiments, the biological sample can be a tissue sample. In some embodiments, the tissue sample can include live cells from a cell culture. In some embodiments, the tissue sample can be a fresh, frozen tissue sample. In some embodiments, the fresh, frozen tissue sample is cryoground into powder. In some embodiments, the biological sample can be live cells on standard tissue culture dishes. In some embodiments, the biological sample can be flash, frozen tissues that have been cryoground into powder and placed on tissue culture dishes, pre-chilled on dry ice.

As used herein, a “cell” can refer to either a prokaryotic or eukaryotic cell, optionally obtained from a subject or a commercially available source.

As used herein, “detecting” can refer to a method used to discover, determine, or confirm the existence or presence of a compound and/or substance (e.g., DNA, RNA, a protein, a fluorophore). In some embodiments, a detecting method can be used to detect a detectable label. In some embodiments, detecting can include chemiluminescence or fluorescence techniques. In some embodiments, detecting can include immunological-based methods (e.g., quantitative enzyme-linked immunosorbent assays (ELISA), Western blotting, or dot blotting) wherein antibodies are used to react specifically with entire proteins or specific epitopes of a protein. In some embodiments, detecting can include immunoprecipitation of the protein.

As used herein, “modulating” can refer to modifying, regulating, or altering the effects of receptor or transporter systems in a cell. In some embodiments, modulating cell uptake can include transporting a molecule or compound (e.g., a labeled molecule, neurotransmitter, metabolite, drugs, proteins, or nanoparticles) into a cell. In some embodiments, modulating cell uptake can include efflux of a molecule or compound from a cell.

As used herein, “nucleic acid” is used to include any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2-amino-α-LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

### Methods of Identifying a Cell Uptake Modulator

Provided herein are methods of identifying a cell uptake modulator of a molecule that include (a) contacting a plurality of cells of a cell-containing biological sample with a plurality of gene-editing agents, wherein a gene-editing agent from the plurality of gene-editing agents recognizes and alters a target gene of at least one cell of the plurality of cells; (b) contacting the plurality of cells with a plurality of molecules, wherein at least one molecule of the plurality of molecules is transported into at least one cell of the plurality of cells; and (c) detecting a presence of the at least one molecule in the plurality of cells, thereby identifying the cell uptake modulator of the molecule.

As used herein, the term “cell uptake modulator” can refer to a ligand that modulates or regulates the mechanism of transporting a molecule across the cell membrane either into or out of a cell. In some embodiments, a cell uptake modulator can transport a molecule into the cell. In some embodiments, a cell uptake modulator can transport a molecule out of the cell. In some embodiments, a cell uptake mechanism can be an active process (e.g., pump transport, vesicle transport, exocytosis, endocytosis, pinocytosis, or phagocytosis). In some embodiments, a cell uptake mechanism can be a passive process (e.g., simple diffusion, osmosis, or facilitated diffusion).

### Gene-Editing Agent

In some embodiments, the method can include delivering a plurality of gene-editing agents into a cell. In some embodiments, the plurality of gene-editing agents can include one or more of the same gene-editing agent. In some embodiments, the plurality of gene-editing agents can include one or more of different gene-editing agents. In some embodiments, a gene-editing agent from the plurality of gene-editing agents comprises a single guide RNA (sgRNA) corresponding to a gene from a gene library, wherein the plurality of gene-editing agents comprises different gene-editing agents comprising different sgRNAs from the gene library.

In some embodiments, the delivering comprises lipofection. In some embodiments, the delivering comprises a virus-based delivery. In some embodiments, the virus-based delivery comprises adeno-associated virus or lentivirus. In some embodiments, the gene-editing agent is a lentivirus. In some embodiments, the gene-editing agent comprises CRISPR/Cas9 components. In some embodiments, the gene-editing agent comprises a single guide RNA (sgRNA), wherein the sgRNA is targeted to the target gene of a cell of the biological sample.

As used herein, the term “gene-editing agent” can refer to an agent that allows for changing the DNA or RNA (e.g., mRNA) in the genome. In some embodiments, gene-editing can include insertion, deletion, modification, or replacement of the DNA or RNA. In some embodiments, a gene-editing agent can include a nuclease-based gene editing platform. In some embodiments, a gene-editing agent can include zinc finger nucleases (ZFNs), transcription activator-like effector nucleases (TALENs), engineered meganucleases, or a clustered regularly interspaced short palindromic repeats (CRISPR) system. In some embodiments, a gene-editing agent can include RNA interference (e.g., short hairpin RNA (shRNA), small interfering RNA (siRNA), antisense oligonucleotide (ASO), or microRNA mimics). In some embodiments, the gene-editing agent can include CRISPR components. For example, in some embodiments, CRISPR components can include, but are not limited to, a guide RNA and a CRISPR-associated endonuclease (Cas protein). In some embodiments, the gene-editing agent can include a guide RNA (e.g., gRNA or sgRNA) and a CRISPR-associated endonuclease (Cas protein). In some embodiments, the gene-editing agent comprises shRNAs, siRNAs, ASOs, or microRNa mimics.

As used herein, the term “CRISPR” refers to a technique of sequence specific genetic manipulation relying on the clustered regularly interspaced short palindromic repeats pathway, which unlike RNA interference regulates gene expression at a transcriptional level. The term “gRNA” or “guide RNA” refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014;32(12):1262-7 and Graham, D., et al. Genome Biol. 2015; 16: 260. The term “Single guide RNA” or “sgRNA” is a specific type of gRNA that combines tracrRNA (transactivating RNA), which binds to Cas9 to activate the complex to create the necessary strand breaks, and crRNA (CRISPR RNA), comprising complimentary nucleotides to the tracrRNA, into a single RNA construct. Exemplary methods of employing the CRISPR technique are described in WO 2017/091630, which is incorporated by reference in its entirety. Non-limiting aspects of CRISPR/Cas9 screening with a gene library containing sgRNAs are described in Wang et al., Science (2014); 343(6166):80-4, WO 2018/005691, and WO 2016/196805, the entire contents of each of which are incorporated herein by reference.

In some embodiments, a single guide RNA can recognize a target gene (e.g., a target RNA), for example, by hybridizing to the target gene. In some embodiments, the single guide RNA comprises a sequence that is complementary to the target gene. In some embodiments, the sgRNA can include one or more modified nucleotides. In some embodiments, the sgRNA has a length that is about 10 nt (e.g., about 20 nt, about 30 nt, about 40 nt, about 50 nt, about 60 nt, about 70 nt, about 80 nt, about 90 nt, about 100 nt, about 120 nt, about 140 nt, about 160 nt, about 180 nt, about 200 nt, about 300 nt, about 400 nt, about 500 nt, about 600 nt, about 700 nt, about 800 nt, about 900 nt, about 1000 nt, or about 2000 nt).

In some embodiments, a single guide RNA can recognize a variety of target genes. In some embodiments, a single guide RNA can recognize a variety of RNA targets. For example, a target RNA can be messenger RNA (mRNA), ribosomal RNA (rRNA), signal recognition particle RNA (SRP RNA), transfer RNA (tRNA), small nuclear RNA (snRNA), small nucleolar RNA (snoRNA), antisense RNA (aRNA), long noncoding RNA (IncRNA), microRNA (miRNA), piwi-interacting RNA (piRNA), small interfering RNA (siRNA), short hairpin RNA (shRNA), retrotransposon RNA, viral genome RNA, or viral noncoding RNA. In some embodiments, a target gene can be a gene involved in pathogenesis of conditions such as cancers, neurodegeneration, cutaneous conditions, endocrine conditions, intestinal diseases, infectious conditions, neurological conditions, liver diseases, heart disorders, or autoimmune diseases. In some embodiments, a target gene can be a therapeutic target for conditions such as cancers, neurodegeneration, cutaneous conditions, endocrine conditions, intestinal diseases, infectious conditions, neurological conditions, liver diseases, heart disorders, or autoimmune diseases.

### DNA-Polymer Amphiphile Nanoparticles (DPANPs)

In some embodiments, a method described herein comprises contacting a plurality of cells with a plurality of molecules, wherein the molecule is a DNA polymer micelle. As used herein, a “DNA polymer micelle” refers to a molecule that can be arranged in a spherical form in an aqueous solution, wherein the DNA polymer micelle includes an aggregate of amphiphiles that have a nonpolar hydrophobic region and a polar hydrophilic region. In some embodiments, a DNA polymer micelle comprises a plurality of amphiphilic compounds, wherein an amphiphilic compound of the plurality of amphiphilic compounds comprises a hydrophobic polymer, a DNA sequence, and a detectable label.

In some embodiments, the DNA sequence comprises a single-stranded DNA sequence. In some embodiments, the DNA sequence comprises about 20 nucleotides. In some embodiments, the DNA sequence comprises about 30 nucleotides. In some embodiments, the DNA sequence comprises about 40 nucleotides. In some embodiments, the DNA sequence comprises about 10 to about 50 (e.g., about 15 to about 50, about 20 to about 50, about 25 to about 50, about 30 to about 50, about 35 to about 50, about 40 to about 50, about 45 to about 50, about 10 to about 45, about 15 to about 45, about 20 to about 45, about 25 to about 45, about 30 to about 45, about 35 to about 45, about 40 to about 45, about 10 to about 40, about 15 to about 40, about 20 to about 40, about 25 to about 40, about 30 to about 40, about 35 to about 40, about 10 to about 35, about 15 to about 35, about 20 to about 35, about 25 to about 35, about 30 to about 35, about 10 to about 30, about 15 to about 30, about 20 to about 30, about 25 to about 30, about 10 to about 25, about 15 to about 25, about 20 to about 25, about 10 to about 20, about 15 to about 20, or about 10 to about 15) nucleotides.

In some embodiments, the DNA sequence comprises a spacer sequence. In some embodiments, the spacer sequence comprises a thymine (T) nucleotide and/or an adenine (A) nucleotide. In some embodiments, the DNA sequence is complementary to a sequence that is not expressed in mammalian cells (e.g., GFP).

In some embodiments, the DNA sequence is covalently linked to the hydrophobic polymer. In some embodiments, the hydrophobic polymer can be a Ring-opening metathesis polymerization (ROMP) polymer.

In some embodiments, the amphiphilic compound includes a detectable label. As used herein, a “detectable label” refer to a directly or indirectly detectable moiety that is associated with (e.g., conjugated to) a molecule to be detected. The detectable label can be directly detectable by itself (e.g., radioisotope labels or fluorescent labels) or, in the case of an enzymatic label, can be indirectly detectable, e.g., by catalyzing chemical alterations of a chemical substrate compound or composition, which chemical substrate compound or composition is directly detectable. Detectable labels can be suitable for small scale detection and/or suitable for high-throughput screening. For example, suitable detectable labels can include, but are not limited to, radioisotopes, fluorophores, chemiluminescent compounds, bioluminescent compounds, and dyes.

In some embodiments, a detectable label can be attached to a composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA / AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP / GFP FRET, BOBO™-1 / BO-PRO™-1, BOBO™-3 / BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP / YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF® -97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2 / BCECF, Fura Red™ (high calcium), Fura Red™ / Fluo-3, GeneBLAzer™ (CCF2), GFP Red Shifted (rsGFP), GFP Wild Type, GFP / BFP FRET, GFP / DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1 / JO-PRO™-1, LDS 751 (+DNA), LDS 751 (+RNA), LOLO™-1 / LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1 / PO-PRO™-1, POPO™-3 / PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red) , Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, Spectrum Green® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red® / Texas Red®-X, Texas Red®-X (NHS Ester), Thiadicarbocyanine, Thiazole Orange, TOTO®-1 / TO-PRO®-1, TOTO®-3 / TO-PRO®-3, TO-PRO®-5, Tri-color (PE-Cy5), TRITC (Tetramethylrhodamine), TruRed (PerCP-Cy5.5), WW 781, X-Rhodamine (XRITC) , Y66F, Y66H, Y66W, YFP (Yellow Fluorescent Protein), YOYO®-1 / YO-PRO®-1, YOYO®-3 / YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800 CW (NHS Ester), WellRED D4 Dye, WellRED D3 Dye, WellRED D2 Dye, Lightcycler® 640 (NHS Ester), and Dy 750 (NHS Ester). In some embodiments, the fluorophore is Cy5.

In some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate chemical substrates (e.g., an oxidizing reagent plus a chemiluminescent compound). Non-limiting examples of chemiluminescent compound families include 2,3-dihydro-l,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and - methoxy substituents, oxalates such as oxalyl active esters, p-nitrophenyl, N-alkyl acridinum esters, luciferins, lucigenins, or acridinium esters.

In some embodiments, the detectable label is located at the 3′ end of the DNA sequence. In some embodiments, the detectable label is conjugated to the DNA sequence via a phosphodiester linkage.

In some embodiments, a DNA polymer micelle comprises a plurality of amphiphilic compounds. In some embodiments, the DNA polymer micelle comprises about 20 amphiphilic compounds. In some embodiments, the DNA polymer micelle comprises about 10 to about 100 (e.g., about 15 to about 100, about 20 to about 100, about 25 to about 100, about 30 to about 100, about 35 to about 100, about 40 to about 100, about 45 to about 100, about 50 to about 100, about 55 to about 100, about 60 to about 100, about 65 to about 100, about 70 to about 100, about 75 to about 100, about 80 to about 100, about 85 to about 100, about 90 to about 100, about 95 to about 100, about 10 to about 95, about 15 to about 95, about 20 to about 95, about 25 to about 95, about 30 to about 95, about 35 to about 95, about 40 to about 95, about 45 to about 95, about 50 to about 95, about 55 to about 95, about 60 to about 95, about 65 to about 95, about 70 to about 95, about 75 to about 95, about 80 to about 95, about 85 to about 95, about 90 to about 95, about 10 to about 90, about 15 to about 90, about 20 to about 90, about 25 to about 90, about 30 to about 90, about 35 to about 90, about 40 to about 90, about 45 to about 90, about 50 to about 90, about 55 to about 90, about 60 to about 90, about 65 to about 90, about 70 to about 90, about 75 to about 90, about 80 to about 90, about 85 to about 90, about 10 to about 85, about 15 to about 85, about 20 to about 85, about 25 to about 85, about 30 to about 85, about 35 to about 85, about 40 to about 85, about 45 to about 85, about 50 to about 85, about 55 to about 85, about 60 to about 85, about 65 to about 85, about 70 to about 85, about 75 to about 85, about 80 to about 85, about 10 to about 80, about 15 to about 80, about 20 to about 80, about 25 to about 80, about 30 to about 80, about 35 to about 80, about 40 to about 80, about 45 to about 80, about 50 to about 80, about 55 to about 80, about 60 to about 80, about 65 to about 80, about 70 to about 80, about 75 to about 80, about 10 to about 75, about 15 to about 75, about 20 to about 75, about 25 to about 75, about 30 to about 75, about 35 to about 75, about 40 to about 75, about 45 to about 75, about 50 to about 75, about 55 to about 75, about 60 to about 75, about 65 to about 75, about 70 to about 75, about 10 to about 70, about 15 to about 70, about 20 to about 70, about 25 to about 70, about 30 to about 70, about 35 to about 70, about 40 to about 70, about 45 to about 70, about 50 to about 70, about 55 to about 70, about 60 to about 70, about 65 to about 70, about 10 to about 65, about 15 to about 65, about 20 to about 65, about 25 to about 65, about 30 to about 65, about 35 to about 65, about 40 to about 65, about 45 to about 65, about 50 to about 65, about 55 to about 65, about 60 to about 65, about 10 to about 60, about 15 to about 60, about 20 to about 60, about 25 to about 60, about 30 to about 60, about 35 to about 60, about 40 to about 60, about 45 to about 60, about 50 to about 60, about 55 to about 60, about 10 to about 55, about 15 to about 55, about 20 to about 55, about 25 to about 55, about 30 to about 55, about 35 to about 55, about 40 to about 55, about 45 to about 55, about 50 to about 55, about 10 to about 50, about 15 to about 50, about 20 to about 50, about 25 to about 50, about 30 to about 50, about 35 to about 50, about 40 to about 50, about 45 to about 50, about 10 to about 45, about 15 to about 45, about 20 to about 45, about 25 to about 45, about 30 to about 45, about 35 to about 45, about 40 to about 45, about 10 to about 40, about 15 to about 40, about 20 to about 40, about 25 to about 40, about 30 to about 40, about 35 to about 40, about 10 to about 35, about 15 to about 35, about 20 to about 35, about 25 to about 35, about 30 to about 35, about 10 to about 30, about 15 to about 30, about 20 to about 30, about 25 to about 30, about 10 to about 25, about 15 to about 25, about 20 to about 25, about 10 to about 20, about 15 to about 20, or about 10 to about 15) amphiphilic compounds.

In some embodiments, the DNA polymer micelle can further comprise a therapeutic cargo. As used herein, the term “therapeutic cargo” refers to a reagent or substance that is related to the treatment of disease or disorder. For example, a therapeutic cargo can include, but is not limited to, nanoparticles, therapeutic agents, small RNA and oligonucleotides (e.g., miRNA and siRNA), small molecule drugs, DNA and plasmids, proteins (e.g., enzymes and inhibitors), or gene-editing complexes). In some embodiments, the therapeutic cargo comprises a nucleic acid.

### Identifying a Cell Uptake Modulator in a Cell

In some embodiments, a method provided herein includes detecting a presence of at least one molecule in the plurality of cells, and thereby identifying a cell uptake modulator of a molecule. In some embodiments, the detecting further comprises: selecting a molecule uptake-deficient cell from the plurality of cells; isolating an sgRNA fragment from the molecule uptake-deficient cell; and profiling the isolated sgRNA fragment to identify the target gene of the isolated sgRNA fragment, wherein the target gene is the identified cell uptake modulator.

In some embodiments, the method can include detecting an absence of at least one molecule in the plurality of cells. In some embodiments, the method can include detecting a presence and/or an absence of a plurality of molecules. In some embodiments, the method can include discriminating between cells based on a predetermined parameter. In some embodiments, a molecule of the plurality of molecules can include a detectable label. In some embodiments, a molecule can include a plurality of detectable labels. In some embodiments, the plurality of detectable labels can include the same detectable label. In some embodiments, the plurality of detectable labels can include different labels.

In some embodiments, the selecting of a molecule uptake-deficient cell can comprise cell sorting through which a specific cell type is separated from other cells in a biological sample, wherein the cell sorting is based on the physical or biological properties (e.g., size, morphological parameters, viability, or extracellular and intracellular protein expression). In some embodiments, the selecting of a molecule uptake-deficient cell can comprise immunomagnetic cell sorting (MACS). In some embodiments, the selecting of a molecule uptake-deficient cell can comprise using microfluidic devices. In some embodiments, the selecting of a molecule uptake-deficient cell can comprise antibody-based methods of cell separation. In some embodiments, the selecting of a molecule uptake-deficient cell can comprise fluorescence activated cell sorting (FACS).

In some embodiments, the isolating of the sgRNA fragment can include selective enrichment by using an RNA oligonucleotide with a sequence complementary to the sgRNA fragment. In some embodiments, the RNA oligonucleotide can comprises a biotinylated RNA oligonucleotide with a sequence complementary to the sgRNA fragment, wherein the biotinylated RNA oligonucleotide can be selected suing biotinylation-streptavidin affinity using any of a variety of methods known to the field (e.g., streptavidin beads). In some embodiments, the RNA oligonucleotides comprise a sequence complementary to a constant region flanking the sgRNA fragment. In some embodiments, the RNA oligonucleotide comprises SEQ ID NO: 1 and SEQ ID NO: 2. In some embodiments, the RNA oligonucleotide comprises SEQ ID NO: 2 and SEQ ID NO: 3.

SEQ ID NO: 1–- Flanking upstream sequence
GTGGAAAGGACGAAACACCG

SEQ ID NO: 2 - Flanking downstream sequence
GTTTT

SEQ ID NO: 3 - Flanking upstream sequence
CGAAACACCG

In some embodiments, the profiling of the isolated sgRNA fragment includes sequencing of the isolated sgRNA fragment. A wide variety of different sequencing methods can be used to profile the sgRNA fragment. In some embodiments, sequencing of polynucleotides can be performed by various commercial systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification.

Other examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, MS-PET sequencing, and any combinations thereof. In some embodiments, the profiling of the isolated sgRNA fragment includes high-throughput sequencing.

### Identification of a Cell Uptake Mechanism

Described herein are methods for identifying a cell uptake modulator to provide insights into mechanisms of transport of therapeutically relevant materials and molecules, thereby creating opportunities for further therapeutic development.

In some embodiments, a method can identify a cell uptake modulator in a cell, wherein the cell uptake modulator is an effector of DNA polymer micelle uptake into the cell. In some embodiments, the cell uptake modulator can include a direct interactor. In some embodiments, the cell uptake modulator can include an indirect interactor. In some embodiments, the cell uptake modulator can include SLC18B1, SLC26A11, miR-4476, KLRC3, USP17L5, HENMT1, ARMC4, NPTX1, MOS, MSR1, SCARB1, or NTC. In some embodiments, the identified cell uptake modulator is SLC18B1.

Also described herein are methods of delivering a therapeutic cargo into a cell that include (a) contacting a plurality of cells of a cell-containing biological sample with a gene-editing agent, wherein the gene-editing agent recognizes and alters a target gene of at least one cell of the plurality of cells; (b) contacting the plurality of cells with a plurality of molecules, wherein at least one molecule of the plurality of molecules is transported into at least one cell of the plurality of cells, and wherein a molecule of the plurality of molecules comprises the therapeutic cargo; and (c) detecting a presence of the at least one molecule in the plurality of cells, thereby delivering the therapeutic cargo into the cell.

Also provided herein are methods of targeting specific tissue for cellular uptake of a DNA polymer micelle that include (a) contacting a plurality of cells of a cell-containing biological sample with a gene-editing agent, wherein the gene-editing agent recognizes and alters a target gene of at least one cell of the plurality of cells; (b) contacting the plurality of cells with a plurality of molecules, wherein at least one molecule of the plurality of molecules is transported into at least one cell of the plurality of cells; (c) detecting a presence of the at least one molecule in the plurality of cells; (d) selecting a molecule uptake-deficient cell from the plurality of cells; (e) isolating an sgRNA fragment from the molecule uptake-deficient cell; (f) profiling the isolated sgRNA fragment to identify the target gene of the isolated sgRNA fragment, thereby identifying a cell uptake modulator of the molecule; and (g) identifying a specific tissue where the target gene is expressed and thereby targeting the specific tissue to uptake the DNA polymer micelle.

Also provided herein are methods of high-throughput screening for a target gene encoding a cell uptake modulator in a cell that include (a) contacting a plurality of cells of a cell-containing biological sample with a plurality of gene-editing agents, wherein a gene-editing agent from the plurality of gene-editing agents recognizes and alters a target gene of at least one cell of the plurality of cells; (b) contacting the plurality of cells with a plurality of molecules, wherein at least one molecule of the plurality of molecules is transported into at least one cell of the plurality of cells; (c) selecting a population of cells having a modulated phenotype; (d) identifying and characterizing an sgRNA in the population of cells, thereby identifying the target gene encoding the cell uptake modulator.

### EXAMPLES

### Example 1 - Nanomaterial Synthesis

### Polymer Synthesis

Reagents were purchased from commercial sources and used without further purification. The hydrophobic polymer portion of the DNA-polymer amphiphile was prepared via ROMP using a norbornene-phenyl monomer ((N-benzyl)-5-norbornene-exo-2,3-dicarboximide), carboxylic acid chain transfer agent (4,4′-(but-2-ene-1,4-diylbis(oxy))dibenzoic acid), and the ruthenium initiator [(IMesH2)(C5H5N)2(Cl)2Ru=CHPh]. Monomer (500 - 600 mg) and initiator (⅟20 molar ratio) were added to separate, dry Schlenk flasks with stir bars charged with N2 and dissolved in dry CDCl3 (3 mL and 0.5 mL, respectively). Once dissolved, the initiator solution was added to the monomer solution via cannulate with stirring. After 20 minutes, a 50 µL aliquot was removed and set aside for characterization by SEC-MALS. To the remaining reaction, 2 equivalents of chain transfer agent (with respect to initiator) in dry DMF was added and allowed to react for 45 minutes. The polymer was purified by precipitation three times in cold MeOH, then the pellet was redissolved in DCM and centrifuged to remove excess chain transfer agent. The supernatant was concentrated to dryness and run on a column (mobile phase 4% MeOH in DCM) and fractions containing the polymer product were combined and concentrated to dryness. Polymer dispersity and molecular weight were measured using a Phenomenex Phenogel 5 µ 10, 10 k-1000 k, 300 × 7.80 mm (0.05 M LiBr in DMF)) using a Shimadzu LC-AT-VP pump equipped with a multiangle light scattering detector (DAWN-HELIOS: Wyatt Technology), a refractive index detector (Wyatt Optilab T-rEX), and a UV-vis detector (Shimadzu SPD-10 AVP) normalized to a polystyrene standard. Polymer dispersity was measured to be 1.005 ; Mn = 5212 g/mol. Successful cross-metathesis with the chain transfer agent was monitored by 1 H NMR spectroscopy on a Varian Mercury Plus spectrometer (400 MHz) by observing the shift of the alkylidene proton.

### DNA Synthesis

The hydrophilic DNA component of the amphiphile was synthesized using standard phosphoramidite coupling on an Applied Biosciences 394 automated synthesizer with a 1000 Å CPG column (Glen Research) on a 1 µmol scale. Phosphoramidite monomers (bz-dA-CE #10-1000-10, Ac-dC-CE #10-1015-10, dmf-dG-CE #10-1029-10, dT-CE #10-1030-10, Fluorescein-dT-CE #10-1056-90 and Cyanine 5 Phosphoramidite #10-5915-95) were purchased from Glen Research and dissolved in dry solvent according to manufacturer instructions. Synthesis conditions include activator 4,5-Dicyanoimidazole (Glen Reasearch), 0.02 M iodine in THF/pyridine/water (Glen Research) as the oxidizer, 3% Trichloroacetic acid (Glen Research) as the deblocking solution, and capping mixtures THF/Pyridine/Ac2O (Glen Research) and 16% 1-Methylimidazole in THF (Glen Research). A 5′- amino modifier (Glen Research) was coupled onto the end of sequences for subsequent conjugation with the polymer. Aliquots of each sequence (2-5 mg) were cleaved from the solid support and deprotected (concentrated ammonia for 24-36 hr) for analysis. The MMT protecting group of the amino modifier was left intact to act as a drag-tag for HPLC purification. HPLC purified sequences were desalted using C18 resin (Ziptips, Millipore) and confirmed by MALDI-TOF using 2’,4',6′-Trihydroxyacetophenone/ammonium citrate and 3-hydroxypicolinic acid as a matrix.

### Micelle Formation

DNA was then conjugated to polymer on solid support. Polymer (30 mg) was dissolved in 100 µL dry DMF with 5 µL DIPEA. Coupling agent (HATU, 0.9 equivalents with respect to polymer) was added to the solution and allowed to activate for 10 minutes. Concurrently, the MMT protecting group on the 5′-amino terminus of the DNA was deprotected using trichloroacetic acid followed by rinsing and drying the DNA on solid support with DCM and Ar, respectively. The DNA on CPG beads were then added to the activated polymer solution in a microcentrifuge tube and allowed to react at room temperature on a shaker. After two hours, the reaction was centrifuged to pool the CPGs with DNA-polymer conjugates and the supernatant was removed. CPGs were washed three times with 1 mL DMF by centrifugation, then a freshly activated solution of polymer (prepared in identical fashion as above) was added to the washed beads for a second reaction, which was left on a shaker overnight. The conjugated CPG was then washed in the same fashion as described previously, and on the last wash the beads were returned to the manufacturer CPG column and washed by flowing 15 mL DMF, followed by 15 mL DCM through the column to remove any unreacted polymer. After drying under Ar, the CPG beads were transferred to 2 mL plastic vials and cleaved/deprotected with 0.5 mL concentrated ammonia for 24-36 hours. Micelles from the DNA-polymer conjugate are spontaneously formed in the aqueous deprotection solution, and after removing the beads by filtration and washing with H2O, DMSO, and formamide, the filtrate containing micelles and any unreacted DNA was dialyzed into nanopure H2O using 10 k MWCO snakeskin dialysis tubing (Thermo Scientific). Crude micelle solution was then concentrated to 1 mL under reduced pressure, and injected on SEC-FPLC (HiPrep 26/60 Sephacryl S-200 High Resolution packed column with mobile phase of 50 mM Tris pH 8.5 on an Akta purifier (Pharmacia Biotech) running a P-900 pump and a UV-900 UV-Vis multi-wavelength detector) to purify. Pure micelles were then dialyzed into nanopure H2O and concentrated under reduced pressure. Characterization of micelles was performed by denaturing PAGE (15% separating, 7% stacking, 1 ×TBE, 8 M Urea) to confirm purity, and TEM to confirm morphology. TEM samples were prepared by glow-discharging grids (formvar/carbon-coated, 400 mesh copper, Ted Pella) at 20 mA for 90 seconds using a s4 Emitech K350 glow discharge unit followed by treatment with 250 mM CaCl2. After blotting away excess salt solution, 3.5 µL micelle sample was added to the grid and allowed to dry for 10 minutes and then washed by passing 3 drops of glass distilled H2O over the grid followed by 3 drops of 1% w/w uranyl acetate stain, the excess of which was immediately blotted away using filter paper. The sample-loaded grid was then loaded onto the microscope for imaging.

### Cell Culture

HEK293T cells were cultured at 37° C. and 5% CO2 in complete medium (Dulbecco’s Modified Eagle Medium, Life Technologies) supplemented with 10% fetal bovine serum (OmegaScientific), and 1X penicillin/streptomycin (Corning). Cells were maintained in 10 cm petri dishes and passaged at ~75 - 90% confluency every 3-5 days.

### Amplification of Pooled sgRNA Plasmid DNA

To amplify the A sub-pool, 400 ng of pooled plasmid DNA was electroporated into Endura (Lucigen) E. coli (performed as 4 electroporations of 2 ul (100 ng) pooled plasmid DNA into 25 uL cells), 2 mL of recovery buffer (Lucigen) was added, and cells were recovered at 37° C. for 1 hour. Cells were then pooled, plated onto 245 mm bioassay plates, and incubated at 33° C. for ~16 hours. Cells were then scraped and maxiprepped (Qiagen) with a maximum of 450 mg bacteria per column. This same procedure was repeated for the B sub-pool. Colony counting estimated total recovery of 1.44×10⁸ and 9.6×10⁷ colonies for the A and B libraries respectively.

### Preparation and Titer of Pooled Library Lentivirus

To prepare pooled library lentivirus, 6×15 cm plates of HEK293XT cells were grown to 40% confluency and transitioned to Opti-MEM reduced serum media for one hour. For each plate, transfection mix was prepared as one mixture of 2.5 mL Opti-MEM, 124 µL Lipofectamine PLUS reagent (ThermoFisher), 14.4 µg Gecko v2 plasmid DNA, 6.2 µg pMD2.G, and 9.3 µg psPAX2, and a second mixture of 2.5 mL Opti-MEM with 62.2 µL Lipofectamine 2000. The two mixtures were incubated for 5 minutes at room temperature, mixed, incubated for 10 minutes at room temperature, and then the 5 mL mixture was added dropwise to the 15 cm plate. Six plates each were transfected for both the A and B library sub-pools. After 6 hours, media was replaced with standard growth media (DMEM with 10% FBS).

Lentivirus was harvested 56 hours post-transfection by filtering media through 0.45 µm PES filter. Filtered media was then ultracentrifuged at 20,000 rpm for 2 hours at 4° C., and 100× concentrated lentivirus was resuspended in DMEM with 10% FBS, incubated overnight at 4° C., and aliquoted and stored at -80° C.

To test lentiviral titer, 1.5 × 10⁶ HEK293XT cells were plated per well of a 24 well plate in DMEM with 10% FBS supplemented with 8 ug/mL protamine sulfate. After 30 minutes, varying amounts of pooled library lentivirus was added and spinfection was performed by centrifuging plates at 37° C. for 2 hours at 2,000 rpm, after which media was changed. The following day, each well was split into paired wells with and without puromycin. After 48 hours of selection, cell survival was determined by hemocytometer count, and viral amounts equivalent to multiplicity of 0.3 were identified and used for following experiments.

### Pooled CRISPR/Cas9 Screening

For full screen experiments, 24 wells of 3×10⁶ HEK293T were plated in 12 well plates in DMEM supplemented with 10% FBS and 8 µg/mL protamine sulfate. After 30 minute incubation, 3 µL lentivirus (equivalent to 0.3 MOI) was diluted in 100 µL of Opti-MEM and added dropwise to each well. Plates were then centrifuged at 2,000 rpm at 37° C. for 2 hours, after which media was changed to standard DMEM supplemented with 10% FBS. 30 hours post-transduction, cells were passaged into media containing 1 µg/mL Puromycin. Cells were passaged for 10 days maintaining at least 65×10⁶ cells (1000× coverage) per passage. For library preparation experiments, cell pellets were frozen for gDNA extraction and library preparation at the indicated timepoints.

### Micelle Uptake CRISPR/Cas9 Screen

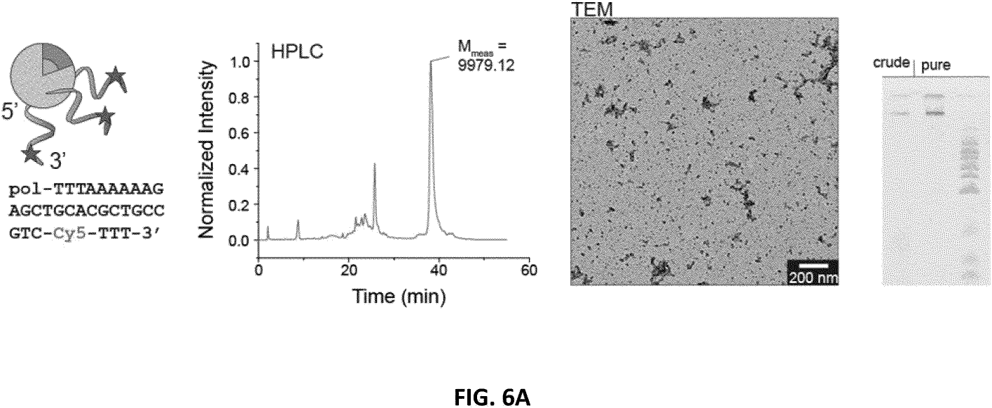

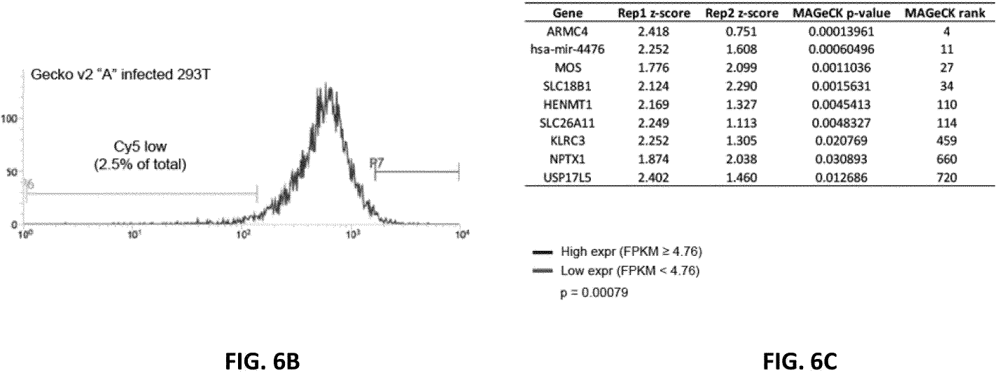

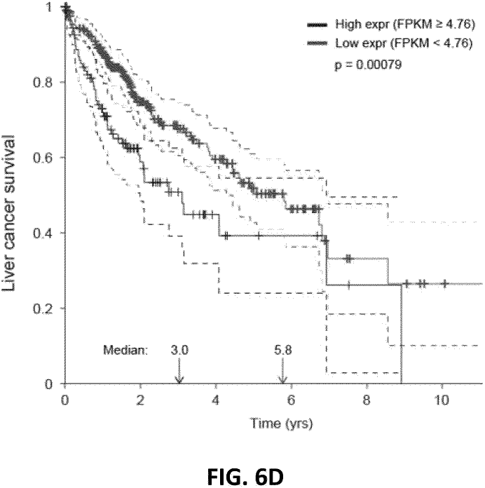

For uptake screening, at 10 days post transduction, cells were treated with 0.1 nM (replicate 1) or 0.2 nM (replicate 2) Cy5-labeled micelles and incubated for 2 hours. To identify uptake-deficient cells, Fluorescent Activated Cell Sorting (FACS) was performed with gates were set to isolate the ~1% (~6.9× 10⁵ cells out of 56 million sorted; replicate 1) and ~3% (1.7× 10⁶ cells out of 96.2 million sorted; replicate 2) of cells with the lowest Cy5 signal (e.g., see FIG. 6A). Final sequenced libraries were prepared from a subset of these samples (Replicate 1: 1.8 µg gDNA equivalent to ~2.7× 10⁵ cells; Replicate 2: 12 µg gDNA equivalent to 1.8×10⁶ cells). As an input, a population of untreated and unsorted cells was selected (Replicate 1: 7.1×10⁶ cells; Replicate 2: 16.1×10⁶ cells).

### Example 2 - Fast Library of Inserts (FLI-seq) Library Preparation

### Probe Generation

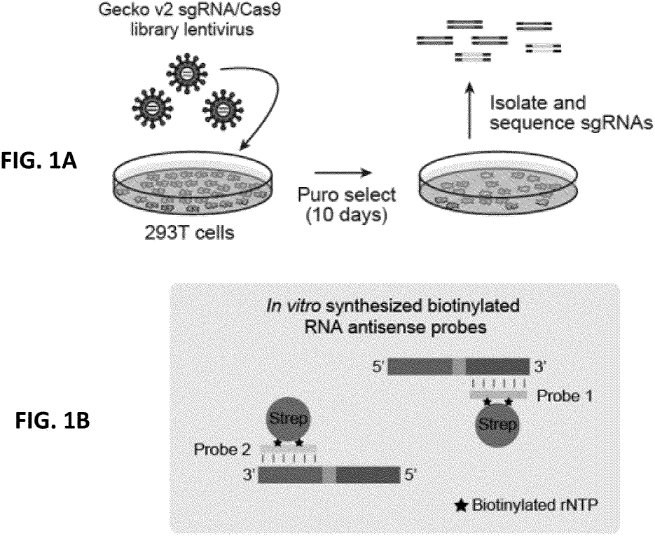

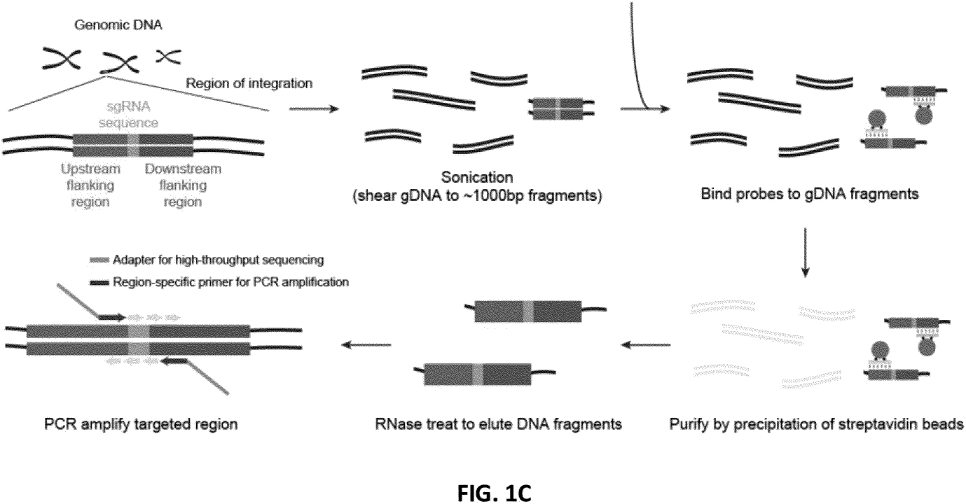

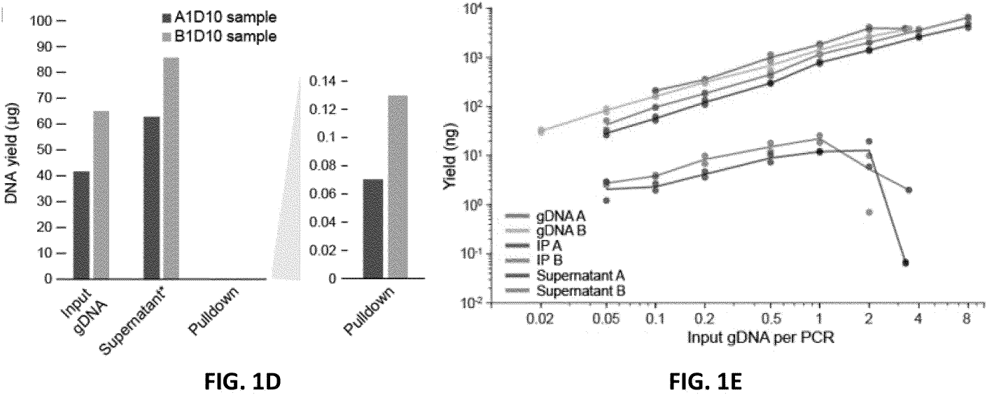

To generate antisense probes for targeted enrichment, two templates with T7 promoter sequences were generated by performing PCR amplification (Q5 Polymerase, NEB) off of GeCKO v2.0 plasmid DNA using H1T7 and H1R primers for probe 1 and primers T2RT7 and T2F for probe 2. Probe 1 hybridizes to the negative strand of upstream sequence, whereas probe 2 hybridizes to the forward strand of the downstream sequence (FIG. 1B). PCR products were cleaned using AMPure XP beads (Beckman Coulter) and manufacturer protocol. In later experiments, amplified PCR products were re-amplified to generate greater probe amounts. Purified dsDNA templates were used as templates for T7 transcription reactions using HiScribe™ T7 High Yield RNA Synthesis Kit (NEB) and bio-16-CTP (Trilink) and bio-16-UTP (Roche) nucleotides at 1:10 ratio to normal nucleotides. After T7 transcription, RNA probes were purified using RNA Clean & Concentrator-25 kit (Zymo Research).

### Enrichment for sgRNA Genomic Sequences and Library Prep

gDNA was isolated using the DNeasy Blood and Tissue Kit (Qiagen), including RNAse A treatment. gDNA was quantified by TapeStation 2200 (Agilent), and then fragmented by sonication (either Q800R2 Sonicator (Qsonica) or Bioruptor Plus (Diagenode)) to 800-1000 bp fragments. After gDNA was denatured, 10% of biotinylated probes (by mass; e.g. 10 µg probes for 100 µg gDNA) were pre-coupled to Streptavidin beads (ThermoFisher) using the manufacturer recommended protocol, and were then hybridized to vector-containing gDNA fragments in 1 × LiCl/Urea Buffer (the final 1 × Buffer contains 25 mM Tris pH 7.4, 5 mM EDTA, 400 mM LiCl, 0.1% NP40, 0.1% SDS, 0.1% sodium deoxycholate, 1M urea) for 3 hours at 60° C. After hybridization, beads were rinsed with 1× LiCl/Urea Buffer at 45° C. for 2-3 minutes. sgRNA-containing gDNA fragments were eluted by digesting RNA probes with Ambion® RNase Cocktail™ (ThermoFisher) and by degrading RNA with NaOH (Sigma). gDNA fragments were purified using the DNA Clean & Concentrator-5 kit (Zymo Research).

Purified gDNA fragments were then PCR amplified (NEBNext Ultra II Q5, NEB) with FLI1F and FLIR primers in PCR1 (denaturation at 98° C. for 30 seconds, followed by 6-9 cycles of denaturation at 98° C. for 12 seconds, annealing at 69° C. for 60 seconds, and extension at 72° C. for 30 seconds, followed by a final elongation at 72° C. for 60 seconds). PCR1 products were purified using AMPure XP beads at 1.6× ratio by volume and manufacturer protocol. After purification, PCR2 was performed with NEBNext Ultra II Q5 (NEB) using standard Illumina indexed primers (e.g. D501 and D701) as follows: denaturation at 98° C. for 30 seconds, followed by 7-8 cycles of denaturation at 98° C. for 10 seconds and annealing and elongation at 72° C. for 40 seconds, followed by a final elongation at 72° C. for 60 seconds). PCR2 products were purified using AMPure XP beads at 1.3× ratio by volume and manufacturer protocol. Libraries were quantified by Tapestation 2200 (Agilent) and sequenced on the HiSeq 2500 or 4000 platforms (Illumina). As these libraries contain identical sequences upstream of the variable sgRNA region, pools submitted for sequencing contained at most 20% FLI-seq libraries, with the remainder of the pool containing high-diversity samples (e.g. standard RNA-seq).

### Analysis of Pooled CRISPR/Cas9 Screens

Sequencing reads were first processed by requiring the presence of flanking upstream (GTGGAAAGGACGAAACACCG (SEQ ID NO: 1)) and downstream (GTTTT (SEQ ID NO: 2)) sequences flanking a 20nt region. These 20nt regions were tallied, and then queried against sgRNA sequences present in the Gecko v2 “A” or “B” libraries. Per-sgRNA Reads Per Million (RPM) were calculated by adding a pseudocount of 1 read and normalizing against the sum of all sequenced reads that contained an exact sequence match to a sgRNA sequence in the queried library. Pair-wise correlations in log2(RPM) were calculated using only sgRNAs with RPM>1 in at least one of the two datasets.

For identification of differentially enriched sgRNAs upon micelle treatment, a pseudocount of one read added to both values and then fold-change was calculated between RPM values in Cy5-low sorted population relative to unsorted. Next, for each sgRNA a z-score was calculated by comparing the fold-change against the distribution of fold-changes observed for 1000 non-targeting sgRNAs. A gene-level z-score was then calculated using the Stouffer’s Z-score method. As an alternative approach, sgRNA counts (as described above) were used as input to the MAGeCK (v0.5.9.3) analysis tool.

To compare against previously published CRISPR/Cas9 screen reproducibility, sgRNA read counts were obtained for the TKO library, including 65 total pair-wise replicate comparisons (3 comparisons in DLD1 cells, 3 timepoints in GBM cells, 2 libraries with 5 timepoints in HCT116 (experiment 1), 2 libraries with 4 timepoints and 3 comparisons per timepoint in HCT116 (experiment 2), 2 libraries with 4 timepoints and 3 comparisons per timepoint in HeLa (excluding T18 for library 1, which was discarded due to abnormal correlation with T15), and 4 timepoints in RPE1 cells. Additionally, sequencing data for screening performed with the Brunello library in A375 cells was obtained. Processing was performed identically as described above by identifying reads containing flanking upstream (CGAAACACCG (SEQ ID NO: 3)) and downstream (GTTT (SEQ ID NO: 2)) sequences and zero mismatch alignment to an sgRNA in the Brunello library. Final read density was calculated by adding a pseudocount of 1 read and normalizing to reads per million based on the total number of zero-mismatch reads in the sample. Pair-wise correlations in log2(RPM) were calculated using only sgRNAs with RPM>1 in at least one of the two datasets.

### Example 3 - Validation of Modulation of Uptake by SLC18B1

### Plasmids

LentiCRISPR v2 plasmids were prepared and amplified. Sequences for the gRNAs were chosen based on: 1) highest enrichment in the pooled screen, or 2) an optimized sequence from the Brunello library. Five non-targeting control guides were also prepared as controls. Insert oligos were purchased from Integrated DNA Technologies (IDT). Post-amplification plasmid sequence was confirmed by Sanger sequencing (Genewiz) of the insert DNA using the LKO0.15’ primer.

### Virus Production

HEK293Ts were seeded in 24-well format at 100 k cells/well one day prior to transfection. Immediately before transfection, medium was removed and replaced with antibiotic-free medium. Packaging plasmid (psPAX2), envelope plasmid (pMD2.G), and transfer plasmid (LentiCRISPRv2) were mixed with Lipofectamine® 2000 (ThermoFisher) in OptiMEM (ThermoFisher) according to the manufacturer protocol and incubated for five minutes prior to adding to wells. A total of 600 ng DNA was transfected per well, with a plasmid ratio of 4:3:1 (wt/wt/wt) packaging:envelope:transfer in a total volume of 50 µL OptiMEM. Virus was harvested at 48 hr and 72 hr post-transfection and filtered through 0.45 µm PVDF syringe filter units (Millipore).

### Transduction

HEK293Ts were seeded in 24-well format at 100 k cells/well one day prior to transduction. Immediately before transduction, medium was removed and replaced with antibiotic-free medium. To each well, 200 µL freshly filtered virus was added. The same transduction procedure was repeated with freshly harvested and filtered virus the following day. At ~ 95% confluency, transduced cells were seeded into 6-well format in 1.5 µg/mL puromycin (Life Technologies) containing medium. Cells were allowed to select in puromycin for 9 days, replacing medium or passaging every 3-4 days.

### Cloning SLC18B1 Knockouts

From the validation knockout populations generated in the previous section, cells were harvested and seeded in 10 cm petri dishes at a density of 800 - 1000 cells/plate. After 3-5 days, five colonies from each knockout were selected and gently removed from the bottom of the dish using a 200 µL pipet and transferred to individual wells of a 96-well plate. At ~ 80% confluency, the cells were passaged. At the next passage, cells were transferred to 48-well plates. mRNA was extracted from the cells and treated with DNaseI (NEB) followed by reverse transcription with Superscript III (Invitrogen) using dT(20) primers according the manufacturer’s instructions. The abundance of target cDNA relative to the housekeeping control GAPDH was measured using qPCR. Cells that demonstrated successful and complete knockout of the target gene were maintained in culture and used in material uptake studies.

### Uptake

Transduced HEK293Ts were seeded in 96-well format at 30 k cells/well one day prior to treatment with micelles. Medium was replaced with 50 µL micelle-containing OptiMEM at a concentration of 0.1 nM (or 0.0 nM for control wells) with respect to micelle (assuming ~ 200 unimers per particle). Treated cells were incubated for 2 hr at 37° C., then the OptiMEM was removed and cells were washed with DPBS and trypsonized. Cells were resuspended in100 µL ice cold DPBS and immediately analyzed by FACS. FACS was performed on a BD FACSCanto and laser settings remained consistent between all wells/runs.

### Example 4 - Simplified Library Preparation for Readout of Pooled Screens

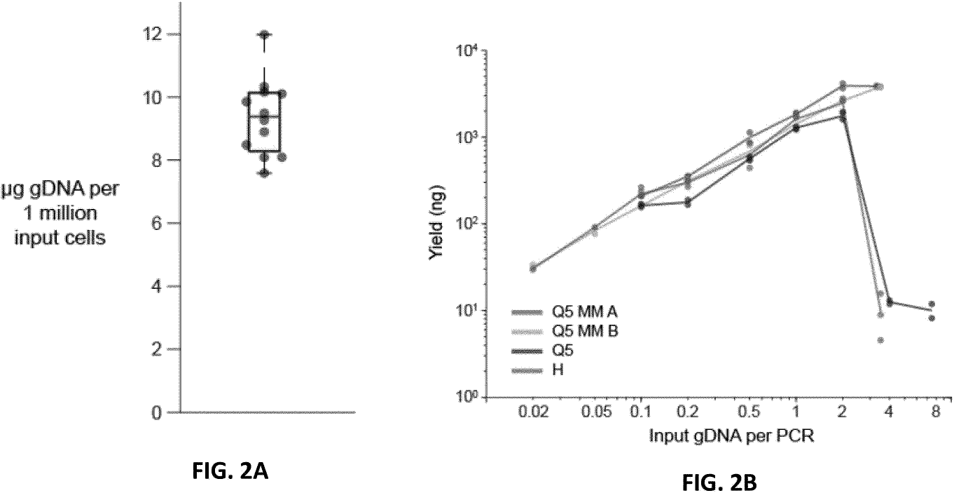

To develop a method for readout of pooled CRISPR/Cas9 screens, a genome-wide CRISPR/Cas9 knockout screen was performed in 293T cells using the published GECKO v2 ‘A’ and ‘B’ libraries, which contains 3 guides targeting each of 19,050 genes for a total of 63,950 and 56,869 unique guides respectively (including 1,000 non-targeting controls (NTCs)) (FIG. 1A). For each replicate, 36 million 293T cells per experiment were transduced with lentivirus at 0.3 multiplicity of infection, after which cells were grown under puromycin selection for 10 days with at least 100x coverage to ensure infection and high knockout efficiency. Genomic DNA (gDNA) was isolated at D0 (immediately after lentiviral spinfection), and 3, 6, and 10 days after infection to assay dropout of essential genes, with an average of ~9.4 µg gDNA recovered per million cells, matching standard expectations for aneuploid HEK293T cells (FIG. 2A). Thus, to maintain 100x coverage of the sgRNA library would require library preparations with ~60 µg gDNA. Using the standard method of simple PCR amplification with targeted primers, inhibition of PCR was observed at ~2 µg of gDNA per PCR (with a 2X master mix formulation showing the least inhibition, but the limited volume available per reaction limits the maximum gDNA in this case) (FIG. 2B). Although this was consistent with standard inhibitory effects of excess DNA, it suggested that proper amplification would require dozens or more separate amplification reactions per sample.

To address this challenge, the Fast Library of Inserts (FLI-seq) approach was developed to remove the majority of unwanted non-sgRNA genomic DNA (FIGS. 1B-1C). However, due to the handling complexity and inefficiency of this approach at large scales, an alternative approach was tested in which pulldown was performed with biotin-labeled antisense RNA oligonucleotides to selectively enrich sgRNA-containing regions relative to overall gDNA. Antisense probes were generated by PCR amplification of a 500 nt constant region flanking the sgRNA sequence, followed by in vitro T7 transcription with biotinylated UTP (FIG. 1B). After incubation, streptavidin pulldown, and washes, it was found that 99.8% of input gDNA remained in the supernatant, with less than 0.2% purified after enrichment (FIG. 1D). However, when PCR was performed with DNA amounts equivalent to equal numbers of input cells, it was observed that the enriched fraction gave similar yields to non-selected gDNA and far greater than supernatant (FIG. 1E). Using values from 0.5 µg-equivalent input material, this corresponds to a ~30-fold increase in amplified yield between enriched and supernatant, indicating a greater than 104-fold enrichment for sgRNA regions in pulldown material (FIG. 1E). Furthermore, the input material was increased to the equivalent of 8 µg total gDNA (=8.5×10⁵) cells per 20 µL PCR reaction without observing noticeable drops in yield (FIG. 1E), indicating that this approach can yield robust amplification with far fewer PCR reactions necessary. It was noted that the enrichment procedure includes an additional column cleanup step, which may explain a large fraction of the ~2.5-fold decreased yield relative to non-selected gDNA; similar loss would be expected from post-PCR restriction digest and gel electrophoresis steps utilized in other protocols.

### Example 5 - Validation of Simplified Library Preparation Reproducibility

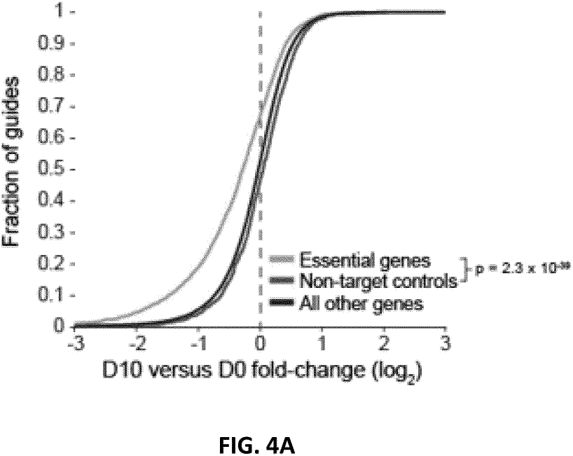

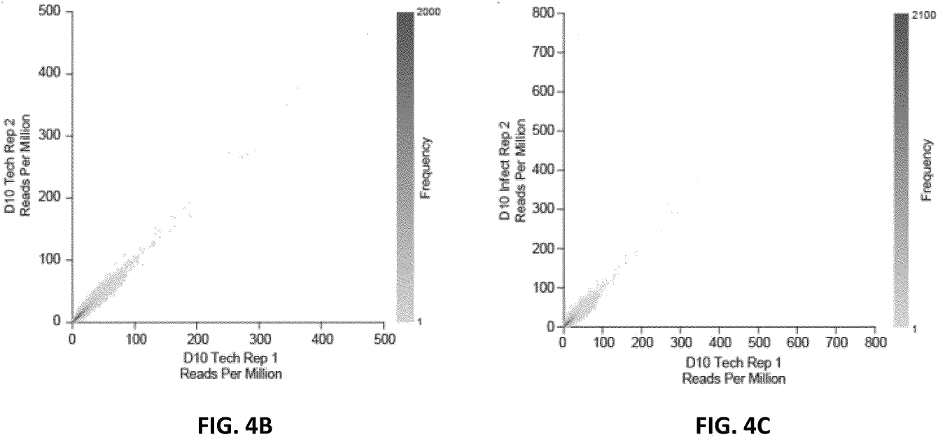

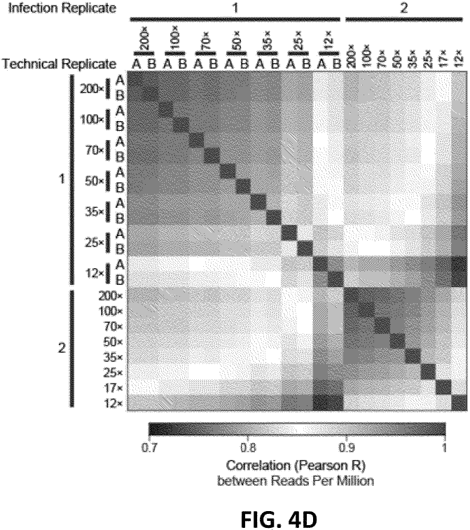

Although this approach significantly decreases the necessary number of PCR reactions and handling complexity, it was critical that the approach properly enriches for sgRNAs that are enriched in the cellular pool and does not introduce high levels of technical irreproducibility. First, to query whether a true signal was recovered, library preparation and high-throughput sequencing of gDNA was performed from D0 and D10 post-infection of HEK293T cells. It was observed that guides targeting essential genes showed significant depletion by D10, confirming successful integration and excision by Cas9 and successful library generation (FIGS. 4A-4D).

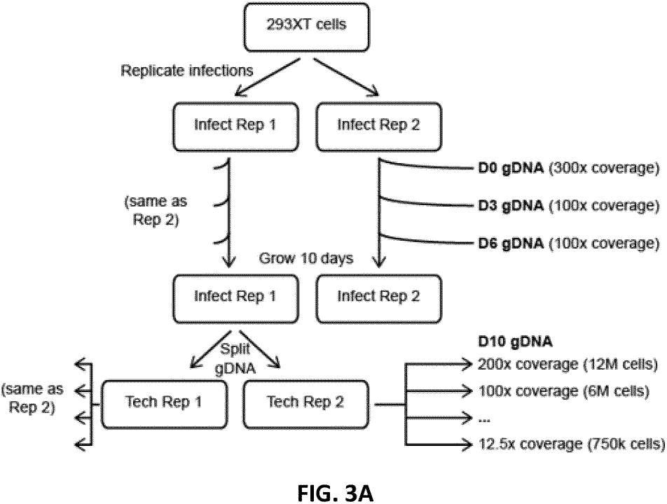

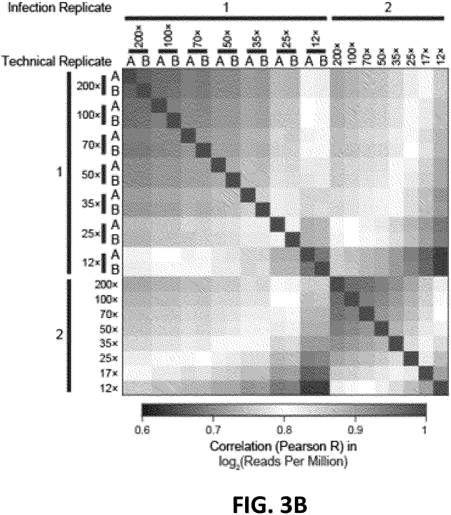

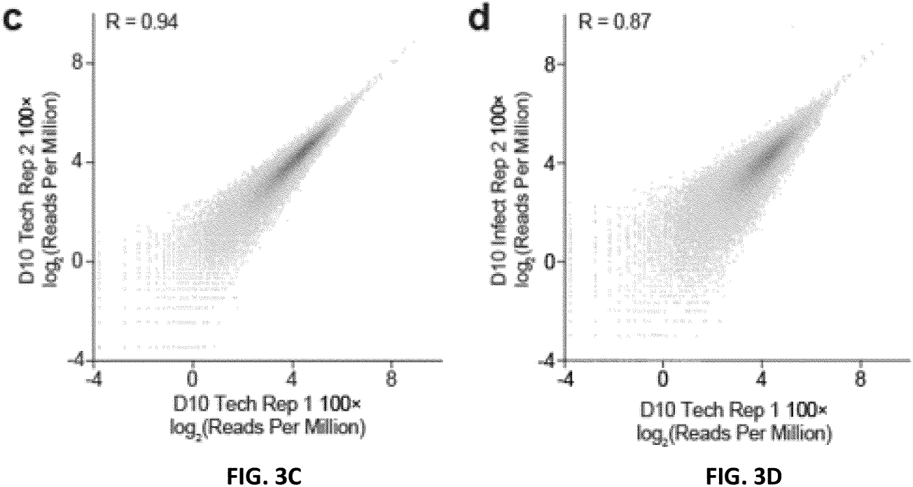

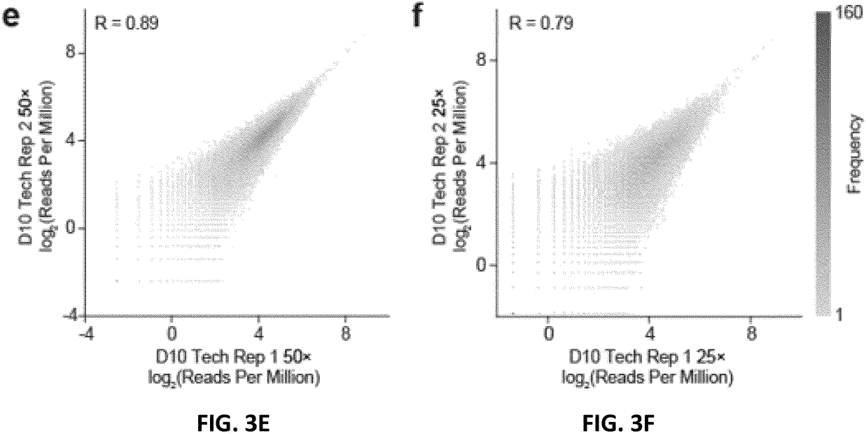

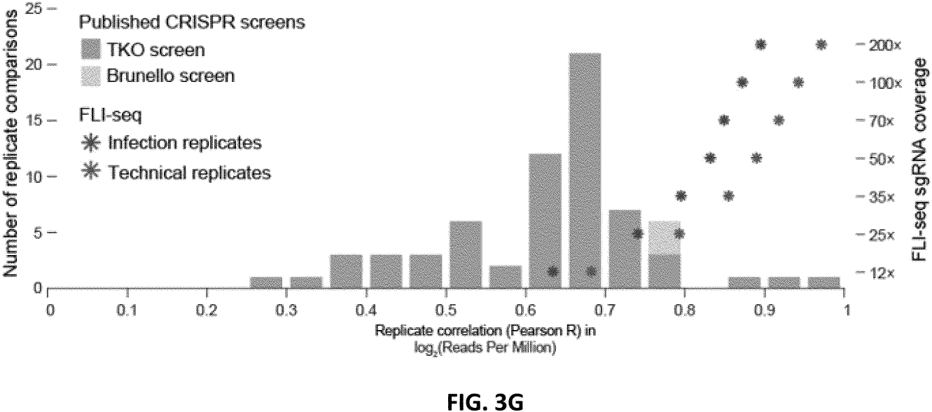

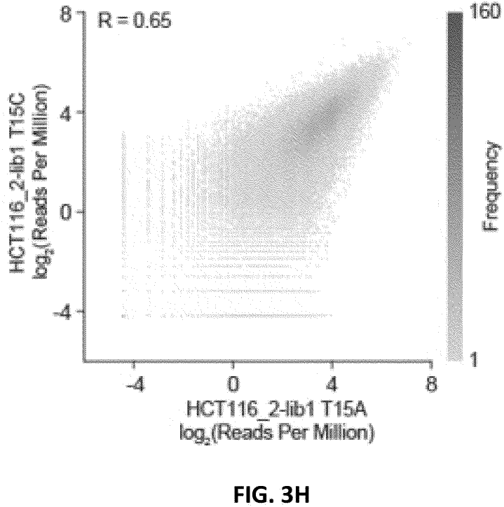

Next, to assay reproducibility, two replicate structures were defined: infection replicates (in which two replicate infections were performed and cells were maintained separately through the entire experiment) and technical replicates (in which after 10 days in culture, isolated genomic DNA was split in half and independently enriched and amplified into library) (FIG. 3A). After processing reads to quantify normalized sgRNA read density, high concordance was observed for technical replicates at 100x coverage (R = 0.94 between log2(RPM)), confirming that the FLI-seq approach reproducibly recovers sgRNAs with low variability (FIGS. 3B-3C). Further, it was observed that infection replicates showed similarly high concordance (R = 0.87), indicating that much of the variability commonly attributed to infection may simply be due to technical library preparation variation (FIG. 3D). Next, the effect of beginning enrichment with variable library coverage was queried by varying the input gDNA amount from 12x to 200x coverage (FIG. 3A). Correlations of 0.88 or above were observed between technical replicates for all samples with at least 50x coverage (~3 million cell input), with 50x technical replicates showing less error than 100x infection replicates (R = 0.89 vs 0.87 respectively) (FIG. 3E). However, significant variability observed when gDNA input was less than 35x, suggesting that bottlenecking remains a significant concern at this low coverage level (FIG. 3F).

To confirm that the FLI-seq approach decreases variability compared to previous approaches, its reproducibility was compared to that observed in previously published CRISPR lethality screens. Considering 65 replicate pairs from TKO library and 3 from Brunello library, a median Pearson correlation of 0.65 (FIGS. 3G-3H) was observed. Indeed, the 90th percentile correlation (R=0.76) was less than that observed with FLI-seq performed with final library readout of only 35x coverage (FIG. 3G). Thus, these results indicate that the FLI-seq method can yield highly reproducible library readouts from CRISPR/Cas9 pooled screens, with higher reproducibility and lower coverage than is frequently used in current procedures.

### Example 6 - Identification of Effectors of DNA Polymer Micelle (DPANP) Uptake

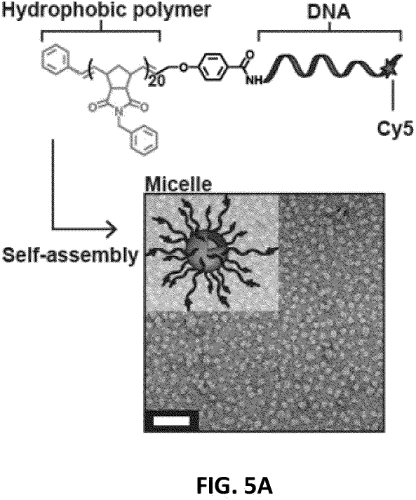

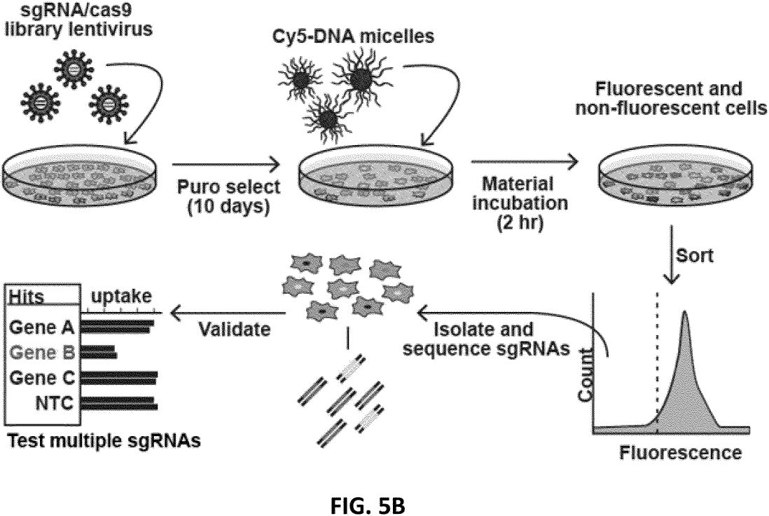

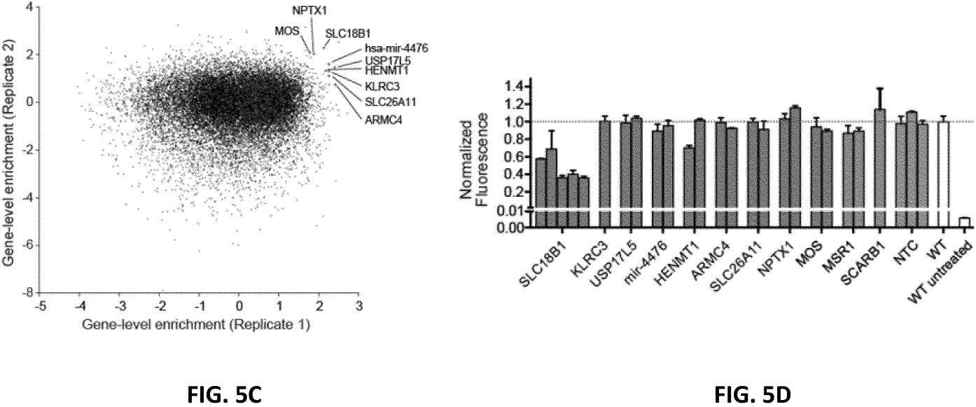

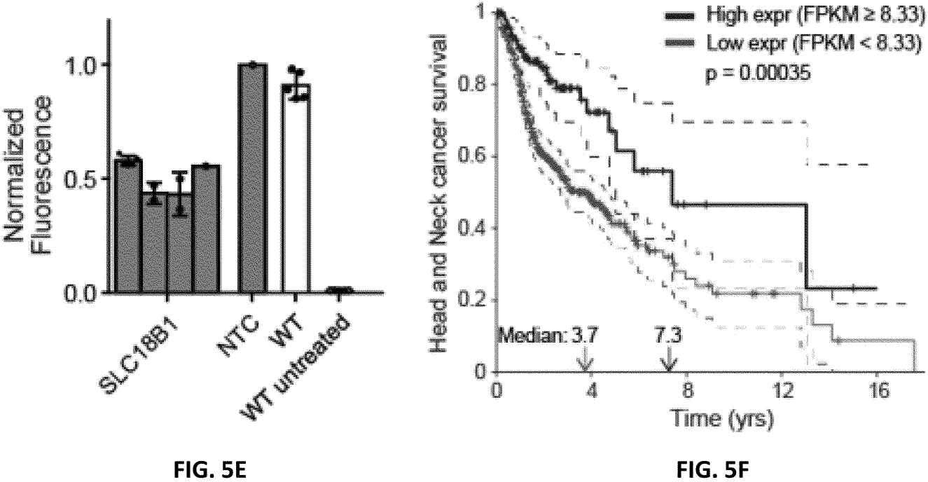

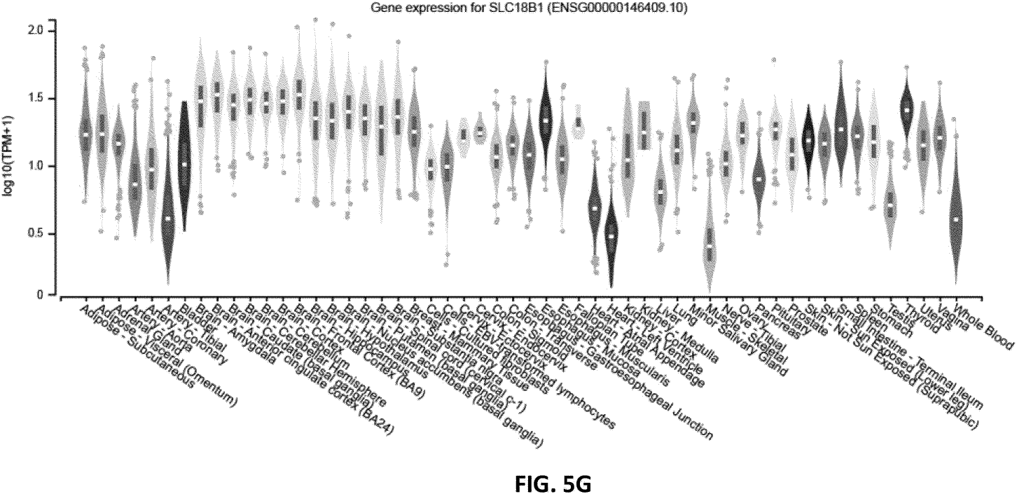

The ability of DPANPs to carry functional nucleic acids into the cytosol of cells and modulate mRNA expression of target genomes without transfection or other helper reagents makes them potential therapeutic nucleic acid carriers, but little is known about how they are able to access the cell interior (FIG. 5A). It was previously shown that incorporation of a cyanine 5 (Cy5) dye into the DNA sequence at the end (3′) of a DPANP structure would enable visualization of micelle uptake into live cells. Regulators of DNA polymer micelle uptake were identified using cellular Cy5 signal as a reporter for micelle uptake, as genetic or other cellular manipulations that decrease uptake will decrease the fluorescence readout (FIG. 5B). To enable this approach, a modified DPANP micelle was generated, wherein the DPANP micelle comprised of a 20-unit hydrophobic ROMP polymer covalently attached to a 30-nucleotide single stranded DNA sequence, with a Cy5 label embedded via phosphodiester linkage within the strand backbone towards the 3′ end (FIG. 6A). The length of the sequence was chosen to be on par with an antisense strand, which is typically in the 20 nt range, and is within facile synthesis range for solid phase phosphonamidite coupling procedures (which typically suffers in yield after 50 or more nts). The DNANP oligo sequence was chosen to be complementary to a sequence not expressed in mammalian cells (GFP), to minimize any potential binding to native sequences in the cell and includes the addition of T and A spacers at the polymer interface.

Two replicate infections of the Gecko ‘A’ library in 293T cells were performed, and after 10-12 days of selection cells were treated with Cy5-labeled micelles followed by fluorescence activated cell sorting (FACS). As cells with the lowest Cy5 signal would contain sgRNA targeted to genes that mediate uptake of the nanomaterials, the lowest 1-3% were selected (Replicate 1; 694 k out of 56 M total input cells, Replicate 2; 1.71 M out of 96.2 M total input cells) as the ‘uptake deficient’ Cy5-low population (FIG. 6B). Genomic DNA was extracted from these populations as well as 7 million (Replicate 1) and 16 million (Replicate 2) unsorted cells as a control (100- and 240-fold library coverage respectively), and high-throughput libraries were generated using the FLI-seq method. A metric for identifying reproducibly enriched targets was developed by calculating a z-score for the fold-enrichment observed for sgRNAs targeting each gene relative to 1000 included non-targeting sgRNAs (FIG. 5C). Using this approach, several potential candidates were identified for effectors of micelle uptake, including both potential direct interactors (solute carrier proteins SLC18B1 and SLC26A11) and indirect effectors (miR-4476) (FIG. 5C). Repeating the analysis using the MAGeCK analysis pipeline revealed generally similar enrichments, including identification of SLC18B1 as among the most significant candidates (FIG. 6C). It was noted that although the screen was designed to identify knockouts with near-complete loss of uptake, a later observation of more modest uptake deficiencies upon individual factor knockout may explain the overall low reproducibility observed in the genome-wide screen (FIG. 5C).

### Example 7 - SLC18B1 Influences DNA Polymer Micelle Uptake

Next, knockout cell populations were prepared for the top candidates to verify their respective influence on uptake. Two separate gRNAs were chosen per gene to prepare knockouts for validation: the most highly enriched guide from the uptake screen (GeCKO V2 ‘A’ library) and, when available, an independent guide from the more recently optimized Brunello human sgRNA library, which was not included in the initial screen. In cases where the Brunello guide was not available, the second most enriched guide from the screen was included. In addition to candidate hits from the screen, knockouts were prepared for two scavenger receptor proteins, MSR1 (rank 7976) and SCARB1 (rank 1904). These receptor proteins have been implicated previously in uptake of similarly structured nanoparticles by other methods, but did not appear to have significant influence in the screen. After transduction and selection for infection, cells were incubated with micelles and fluorescence of each knockout was determined by flow cytometry. During this analysis, it became apparent that the highest ranked gene candidate, SLC18B1, displayed a significant (ca. 40-60%) reduction in fluorescence after material incubation. The other genes examined, as well as the genes implicated in the literature, showed no significant effect (FIG. 5D).

To further confirm the effect of SLC18B1, an additional three guides were tested (including guides not included in the original screen) which also showed a significant reduction in uptake compared to wildtype and non-targeting guide controls (FIG. 5D). Next, to account for potential heterogeneity of expression within the SLC18B1 knockout populations, clones were selected for four of the SLC18B1 guides and validated SLC18B1 mutation by targeted sequencing. Again, it was observed that upon micelle treatment that these cells demonstrated a reduction in fluorescence post-incubation consistent with the pooled SLC18B1 knockouts (FIG. 5E).

As one of the key advantages of DPANPs is to carry nucleic acids or other cargos into cells, the ability to differentially target cells based on SLC18B1 expression would present a potential avenue for future development of targeted therapies. To explore the potential for such approaches, it was queried whether altered SLC18B1 expression was associated with disease progression in the Cancer Genome Atlas (TGCA) resource. Notably, it was observed that higher expression of SLC18B1 served as a significant positive prognostic marker for head and neck cancer (FIG. 5F) and negative marker for liver cancer (FIG. 6C). Considering overall tissue-level expression from the GTEx consortium, SLC18B1 shows particularly higher expression across a variety of brain subregions, with variable expression in other tissues (FIG. 5G). Thus, not only does SLC18B1 represent a novel effector of DPANP uptake, the variable expression suggests that it may serve as an entry point into developing targeted DPANP therapies for different tissues or cell types.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a cell uptake modulator of a molecule, the method comprising:
(a) contacting a plurality of cells of a cell-containing biological sample with a plurality of gene-editing agents, wherein a gene-editing agent from the plurality of gene-editing agents recognizes and alters a target gene of at least one cell of the plurality of cells;
(b) contacting the plurality of cells with a plurality of molecules, wherein at least one molecule of the plurality of molecules is transported into at least one cell of the plurality of cells; and
(c) detecting a presence of the at least one molecule in the plurality of cells, thereby identifying the cell uptake modulator of the molecule.

2. The method of claim 1, wherein the gene-editing agent is a lentivirus.

3. The method of claim 1, wherein the gene-editing agent comprises CRISPR/Cas9 components.

4. The method of claim 1, wherein the gene-editing agent from the plurality of gene-editing agents comprises a single guide RNA (sgRNA) corresponding to a gene from a gene library, and wherein the plurality of gene-editing agents comprises different gene-editing agents comprising different sgRNAs from the gene library.

5. The method of claim 1, wherein the molecule is a DNA polymer micelle.

6. The method of claim 5, wherein the DNA polymer micelle comprises a plurality of amphiphilic compounds, wherein the amphiphilic compound of the plurality of amphiphilic compounds comprises a hydrophobic polymer, a DNA sequence, and a detectable label.

7. The method of claim 6, wherein the detectable label is a fluorophore.

8. The method of claim 7, wherein the fluorophore is Cy5.

9. The method of claim 6, wherein the detectable label is located at the 3′ end of the DNA sequence.

10. The method of claim 6, wherein the DNA sequence comprises about 30 nucleotides.

11. The method of claim 6, wherein the DNA sequence comprises a spacer sequence.

12. The method of claim 5, wherein the DNA polymer micelle comprises about 20 amphiphilic compounds.

13. The method of claim 5, wherein the DNA polymer micelle further comprises a therapeutic cargo.

14. The method of claim 13, wherein the therapeutic cargo comprises a nucleic acid.

15. The method of claim 1, wherein the detecting step (c) further comprises: selecting a molecule uptake-deficient cell from the plurality of cells; isolating an sgRNA fragment from the molecule uptake-deficient cell; and profiling the isolated sgRNA fragment to identify the target gene of the isolated sgRNA fragment, wherein the target gene is the identified cell uptake modulator.

16. The method of claim 15, wherein the profiling comprises sequencing of the isolated sgRNA fragment.

17. The method of claim 16, wherein the sequencing comprises high-throughput sequencing.

18. The method of claim 15, wherein the identified cell uptake modulator is SLC18B1.

19. The method of claim 1, wherein the cell-containing biological sample comprises a tissue sample.

20. The method of claim 1, wherein the cell-containing biological sample comprises live cells from a cell culture.


SEQUENCE LISTING (3 sequences)

SEQ ID NO: 1 - RNA, 20 nt, synthetic construct
  source 1..20: mol_type = other RNA
gtggaaaggacgaaacaccg

SEQ ID NO: 2 - skipped

SEQ ID NO: 3 - RNA, 10 nt, synthetic construct
  source 1..10: mol_type = other RNA
cgaaacaccg